(19) United States
(12) Patent Application Publication
(10) Pub. No.: US 2023/0088449 A1
(43) Pub. Date: Mar. 23, 2023

(54) Carbon Nanotube Coating for Increasing ECG Electrode Conductivity via Capillary Action of Sweat

(71) Applicant: Google LLC, Mountain View, CA (US)

(72) Inventor: Yogesh Verma, Pleasanton, CA (US)

(21) Appl. No.: 17/480,842

(22) Filed: Sep. 21, 2021

(51) Int. Cl. A61B 5/263; A61B 5/00; A61B 5/28

(52) U.S. Cl. CPC A61B 5/263; A61B 5/4266; A61B 5/7264; A61B 5/28

(57) ABSTRACT

A user device containing carbon nanotubes coatings for increasing ECG electrode conductivity while maintaining visually dark electrodes in wearable devices. The carbon nanotubes can also allow for the carbon nanotube coated electrode to increase or cause a capillary action of sweat to increase conductivity of the sweat and signal strength from the skin.

### BACKGROUND

Electrocardiography (ECG or EKG) is the process of producing an electrocardiogram. The electrocardiogram can be a graph of voltage versus time related to the electrical activity of the heart which can be estimated or derived through the use of electrodes placed on the skin of a user. ECG features are ubiquitous on wearable devices, such as smartwatches.

Chromium-Nitride (Cr_{X}N_{Y}), which is a combination of chromium and nitrogen, is often used to form the electrode of a wearable device through vacuum deposition coating. The stoichiometric ratio of chromium and nitrogen in Cr_{X}N_{Y} can be chosen based on hardness, appearance, conductivity, or other requirements related to the wearable device. While Cr_{X}N_{Y} is often an electrode or electrode coating choice due to its darker color appearance, the higher nitrogen content included to increase the darkness lowers the conductivity of the electrode and in turn decreases the signal strength obtained from the skin via the electrode.

### SUMMARY

The present disclosure provides for methods to use carbon nanotubes coatings for increasing ECG electrode conductivity while maintaining strict requirements for visually dark electrodes in wearable devices. The disclosure also provides for a carbon nanotube coated electrode which can increase the capillary action of sweat to increase conductivity and signal strength from the skin.

Aspects of the disclosed technology include a wearable device. The wearable device can include a first surface or a first back surface with a substantially lateral plane. Carbon nanotubes can extend substantially orthogonally to the first back surface or first surface. The wearable device can contain a memory with instructions, and the instructions can be configured to analyze health signals received by the wearable device from a first ECG sensor or ECG electrode. The wearable device can contain a processor to analyze the health signals and provide summary information and a display to display the summary information. The wearable device can contain a health sensor located parallel to the lateral plane of the first back surface to analyze health information of a user. The health sensor can be an ECG electrode or ECG sensor. Carbon fibers can extend lengthwise orthogonally to the ECG electrode. The carbon fibers can be configured to wick sweat from the skin of a user towards the ECG electrode or ECG sensor through capillary action. The device can contain a sweat sensor, the sweat sensor configured to analyze analytes or metabolites contained within the sweat of a user. The carbon fibers can be are configured to wick swat from the skin of a user towards the sweat sensor. An ECG algorithm can be used to analyze the ECG signals. The ECG algorithm can be a machine learning algorithm. The density of the carbon fibers can be selected to increase the wicking of sweat. The radius of the carbon fiber from the carbon fibers is selected to increase the wicking of sweat or a capillary action. The relative height and radius of the carbon fiber from the carbon fibers can be selected to increase the wicking of sweat or to increase a capillary action.

Aspects of the disclosed technology can include a method comprising receiving an ECG signal from an ECG electrode, the ECG electrode configured to contact skin of a user, and the ECG electrode containing carbon fibers extending outwards from the ECG electrode analyzing the ECG signal to determine health information of the user; and displaying, to the user, health information obtained from the analyzed ECG signal. The ECG signal can be enhanced due to the presence of carbon fibers. An ECG algorithm can be used to analyze the ECG signals. The health information can be analyzed using a machine learning algorithm.

Aspects of the disclosed technology can include a non-transitory computer readable medium containing instructions, the instructions when executed configured to receive an ECG signal from an ECG electrode, the ECG electrode configured to contact skin of a user, and the ECG electrode containing carbon fibers extending outwards from the ECG electrode, analyze the ECG signal to determine health information of the user, and display, to the user, health information obtained from the analyzed ECG signal, wherein the ECG signal has been enhanced due to the presence of carbon fibers.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a graph of electric resistivity within a Chromium-Nitride film as a function of Nitrogen (N₂) content according to aspects of the disclosure.

FIG. 2A is a diagram of a user device according to aspects of this disclosure.

FIG. 2B is a diagram of a cross sectional view of a user device according to aspects of this disclosure.

FIG. 3A is a diagram of a cross sectional view of a user device according to aspects of this disclosure.

FIG. 3B is a perspective view of a user device according to aspects of this disclosure.

FIG. 3C is a top-down view of a user device according to aspects of this disclosure.

FIG. 4 is a diagram of user interfaces according to aspects of this disclosure.

FIG. 5 is a flowchart of an example method according to aspects of this disclosure.

FIGS. 6A and 6B illustrate example displays of summary information.

### DETAILED DESCRIPTION

This disclosure generally relates to the use of carbon nanotube coatings as they relate to the electrodes in wearable devices. As explained herein, the use of carbon nanotubes increases conductivity while maintaining visual properties of darkness. Carbon nanotubes or carbon fibers can be deposited onto a surface of a user device. The carbon nanotubes or carbon fibers can be deposited onto an electrode or sensor of the user device and extend outwards from the surface of the electrode, surface, or sensor.

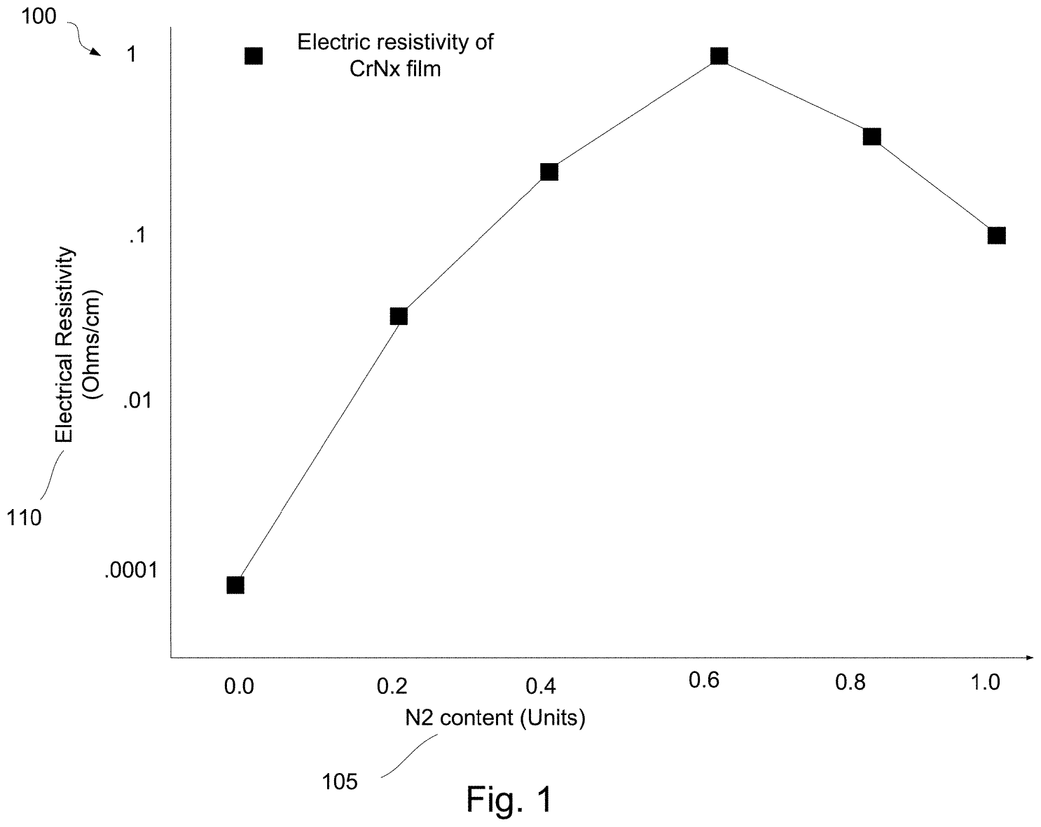

FIG. 1 illustrates a graph 100 of electrical resistivity as a function of Nitrogen (N₂) content according to aspects of the disclosure. The vertical axis 110 indicates the electrical resistivity as measured in Ohms (Ω)/cm. The horizontal axis 105 represents the Nitrogen (N₂) content as molality in a CrNₓ compound, where x represents the proportion of Nitrogen (N) compared to Chromium (Cr). As can be seen from graph 100, an increase in Nitrogen increases the electrical resistivity of the compound by several orders or magnitude. Yet, a threshold amount of nitrogen, such as at levels of x=1, is required to achieve an optically “dark” profile required in wearable devices.

Not illustrated on graph 100 due to the scale is the electrical resistivity profile of carbon fiber. Carbon fiber is a material made up of “fibers,” typically about 5 to 10 micrometers in diameter, and composed mostly or entirely of carbon atoms. The structure of carbon fibers provides for high stiffness, high tensile strength, chemical resistance, high temperature tolerances, and low thermal expansion. The electrical resistivity profile of carbon fiber can vary between 2.0 to 20 micro ohms per meter (μΩ/m), which is several orders of magnitude less than CrNₓ. Carbon fiber is optically “dark” and black in color and meets the dark profile required in wearable devices. Additionally, carbon fiber, due to its hardness, has higher scratch and abrasion resistance as compared to other materials.

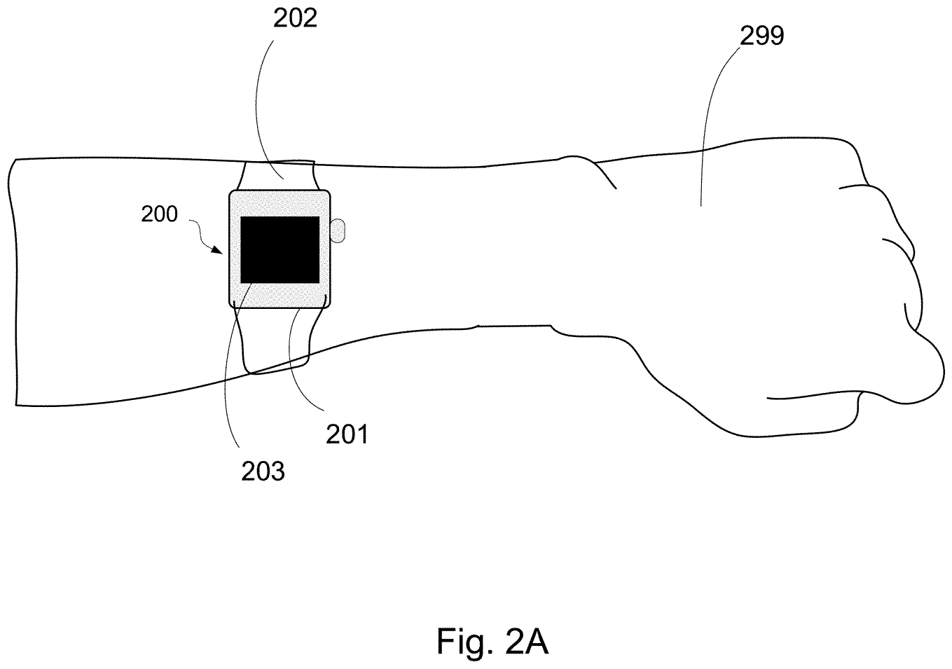

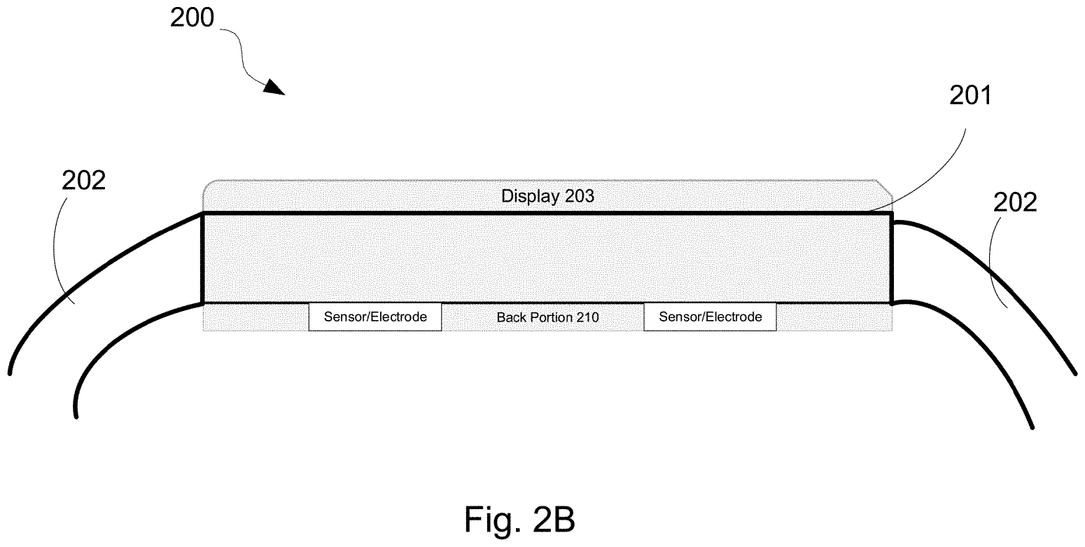

FIG. 2A illustrates a user device, 200, which can be used by a user 299. While FIG. 2 illustrates the user device 200 as a smartwatch, in other examples the user device 200 can be a bracelet, a health sensor, an earbud, headphone, or other wearable device such as a ring, an anklet, necklace, or other piece of jewelry.

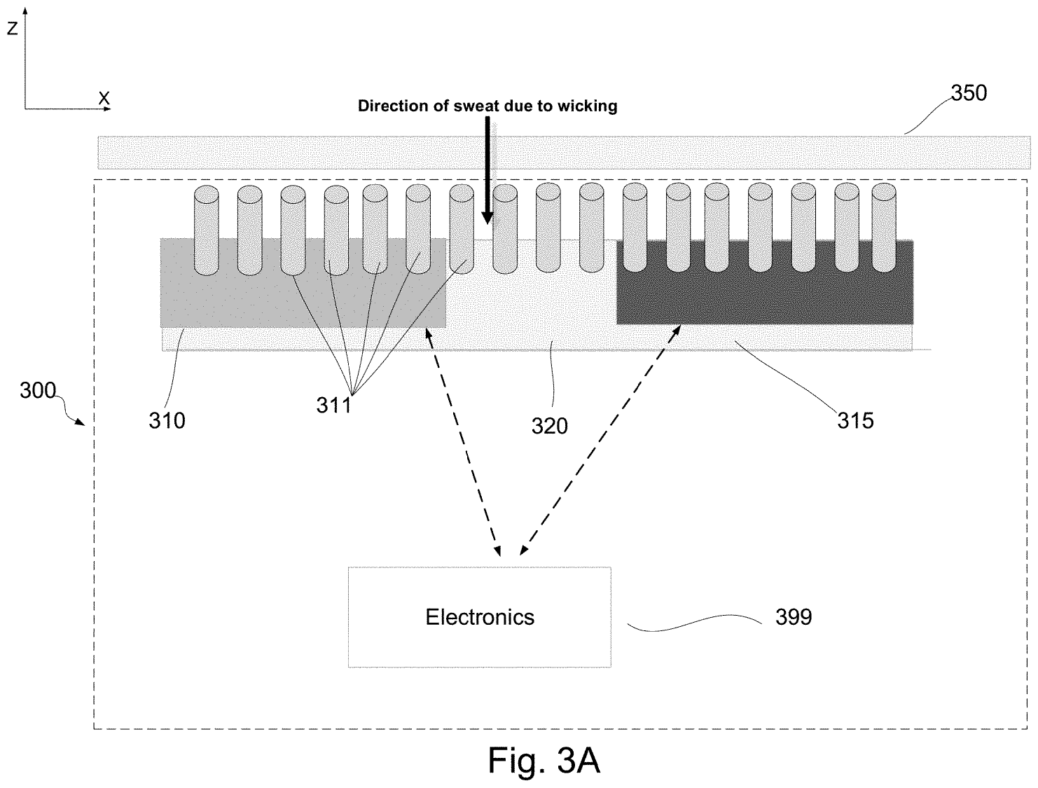

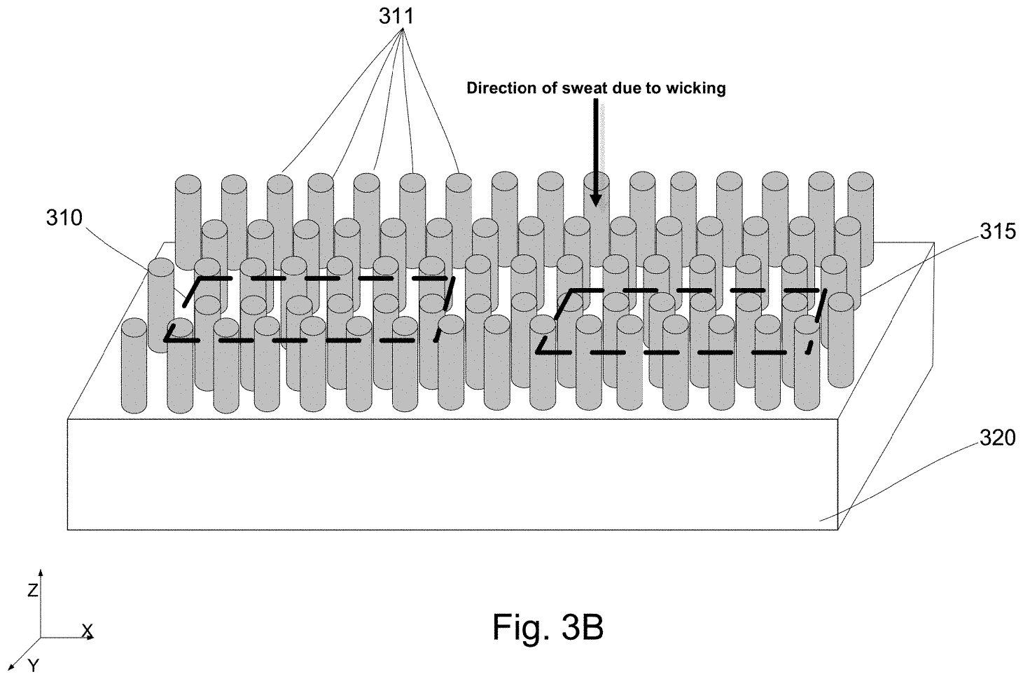

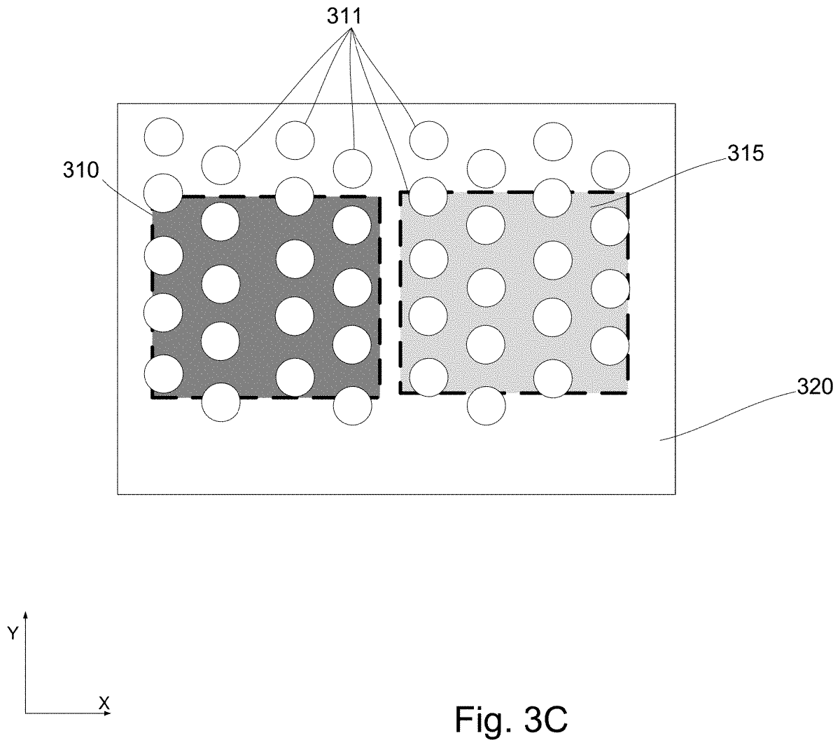

The user device can include a housing 201, and a strap 202. The user device can also include a display 203 and a user interface, for example within the housing 201. The user interface may allow user 299 to interact with information displayed on the display 203 of the user device 200. The user interface can be part of a touchscreen or other device. Additional components which can be included in user device 200 or in housing 201 are further described below with reference to FIGS. 3 to 5. Strap 202 can be a strap to hold the user device on a user, such as one made from metal, leather, cloth, or other material.

FIG. 2B illustrates a cross sectional view of user device 200. Housing 201 can include components such as a back portion 210 configured to contact the skin of user 299 when the user device 200 is worn. The back portion can contain a glass portion which will allow light to pass through the back portion. In other examples, the back portion can include one or more sensors or electrodes. The sensors or electrodes can be used for measuring electrical or other signals from user 299. For instance, the electrodes can include ECG electrodes which are configured to obtain electrical signals from the skin of the user to process ECG data.

In some examples, the electrodes can be formed from a conductive material, such as a metal or metal compound. The electrode can be coated with carbon fiber. In some examples, the electrode can be formed by using a catalyst based chemical vapor deposition (CVD) technique. CVD deposition of carbon nanotubes onto an electrode allows for high scratch resistance, abrasion resistance, while maintaining the high thermal and electrical conductivity properties described above.

FIG. 3 illustrates a cross sectional representation of device 300, which can be used to perform ECG measurements. Device 300 can be a user device, such as a wearable device, similar to user device 200. The device 300 includes a sensor 310 with carbon fibers 311 embedded onto sensor 310. The sensor can capture or generate electrical signals which can be coupled to electronics 399. The electronics 399 can include processing components configured to receive the electrical signals from the sensors and perform one or more functions in response, such as calculating biometric information, providing an output, communicating with a paired device, etc.

Carbon fibers 311 can extend from sensor 310 towards a user's skin 350 when the device 300 is worn. The carbon fibers 311 may be hollow, tubular structures which are formed from mostly carbon atoms, bonded together in crystals, which are aligned parallel to the “long” axis of the carbon fiber. In some examples, the carbon fibers can be formed from functionalized graphene with an sp2-sp3 hybridization orbit. Due to their hollow and tubular structure, carbon fibers 311 can further assist with the “wicking” of human sweat into or between carbon fibers 311. The “wicking” can be a type of capillary action. Capillary action is the process of a liquid flowing in narrow spaces without the assistance of, or even in opposition to, external forces like gravity. The presence of the carbon fibers provides a channel for sweat to enter into. As human sweat contains electrolytes, sweat can further increase the conductivity between human skin and sensor 310, thereby increasing the signal to noise ratio and strength of signal received during measurements on the skin, such as for example, ECG measurements.

Sensors 310 and 315 can be any device, circuitry, or module which can obtain information related to the environment or related to a user device. In some examples sensor 310 can be configured for ECG measurements while sensor 315 can be configured to detect or analyze components of sweat only. For example, sensors 310 or 315 can be a temperature sensor, proximity sensor, accelerometer, infrared sensor, pressure sensor, light sensor, touch sensor, humidity or sweat detection sensor, gyroscope, magnetic sensors, microphones, or tilt sensor. Additionally, sensors 310 and 311 can be any device, circuitry, or module which can be used to observe or determine information related to a health state of a user, such as, for example, ECG or ECG signals, blood pressure, blood oxygen levels, stress, temperature, or other metrics which can be derived from a combination of the exemplary aforementioned metrics. Non-limiting examples of sensors 310 and 315 include photoplethysmography (PPG) sensors or modules, temperature sensors, infra-red sensors, photodiodes, voltage sensors, and the like. The sensors can be digital or analog sensors. Sensors 310 or 315 can contain additional components such as an analog front end, photodetectors, accelerometers, or health sensors, such as photoplethysmography sensors, devices, or circuitry.

In addition, the “wicking” effect can allow for additional sensing applications to be performed by device 300. As sweat also includes other biological or metabolic markers, or markers related to biological conditions of a user, such as for example ascorbic acid, uric acid, metabolites like glucose and lactate, electrolytes such as Sodium or Potassium ions (Na+ and K+), the “wicking” effect of the sweat can be used to transport the sweat to an additional sensor designed for detecting or analyzing such markers, such as sensor 315.

In some examples, the distance between carbon fibers 311 or the density of the carbon fibers can be chosen based on particular use applications or optimized to maximize the “wicking” effect described above. In other examples, the relative density of sensors can be varied according to the desired wicking effect. For example, the density of the carbon fibers can be more or less dense closer to sensor 315. In other examples, the thickness or diameter of the carbon fibers, or the relative thickness to length of the fiber, can also be optimized or chosen to increase the wicking effect. For example, a higher length of fiber to the thickness or radius of a carbon fiber or carbon fiber tube, can lead to a higher capillary action.

Further, certain applications, such as iontophoresis, require the application of an electric current from device 300 onto the skin 350 in order to induce sweating for sensing of markers or other parameters through analysis of the induced sweat. Iontophoresis is a process which can be used for transdermal drug delivery by use of a voltage gradient on the skin of a user. In Iontophoresis, molecules can be transported across a skin through electrophoresis and electroosmosis, and the applied electric field can also increase the permeability of the skin. The use of carbon fiber obviates the use of an electric current on a user the need to induce sweat due to the wicking effect caused by the carbon fibers. Further, as some users have high skin sensitivity and have adverse effects to external current for iontophoresis, analysis of sweat in such individuals can be performed due to the wicking effect of carbon fibers without producing said adverse effects.

FIG. 3B illustrates a perspective view of aspects of device 300. FIG. 3B illustrates a plurality of carbon fibers 311 extending vertically from a surface of device 300. Surface 300 may contain a plurality of sensors or electrodes, such as sensor 310 and 315.

FIG. 3C illustrates a top-down view of aspects of device 300. FIG. 3C illustrates a plurality of carbon fibers 311 which have been deposited or formed on a back portion 300 of device 300 as well as on sensors 310 and 315.

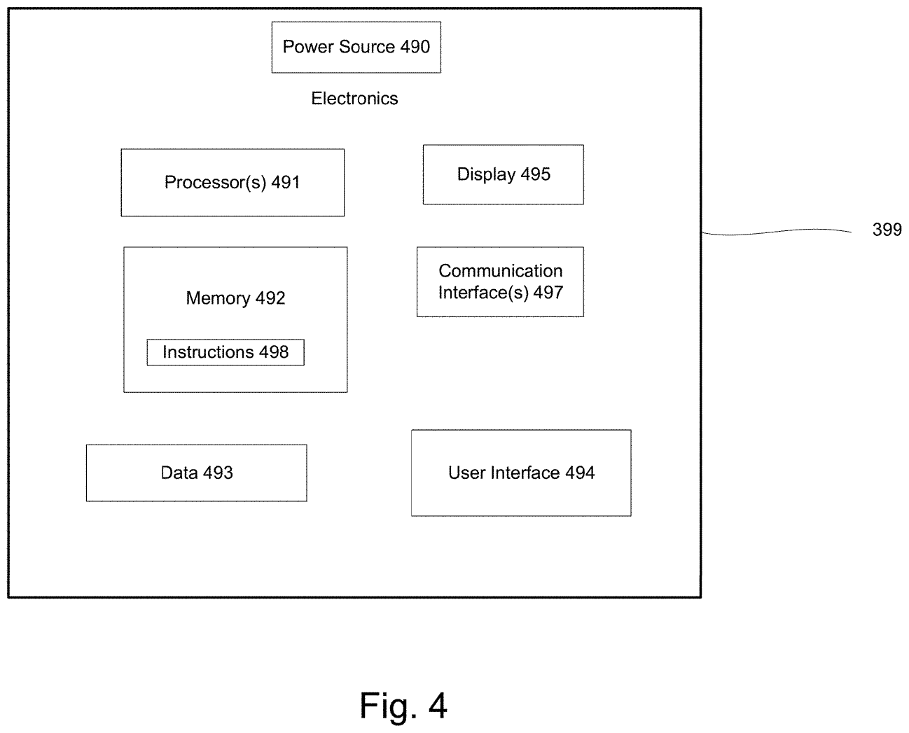

FIG. 4 illustrates aspects of electronics 399. Electronics 399 may contain a power source 490, processor(s) 491, memory 492, data 493, a user interface 494, a display 495, communication interface(s) 497, and instructions 498. The power source may be any suitable power source to generate electricity, such as a battery, a chemical cell, a capacitor, a solar panel, or an inductive charger. Processor(s) 491 may be any conventional processors, such as commercially available microprocessors or application-specific integrated circuits (ASICs); memory, which may store information that is accessible by the processors including instructions that may be executed by the processors, and data. Memory 492 may be of a type of memory operative to store information accessible by the processors, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions and data are stored on different types of media. Data 493 of electronics 499 may be retrieved, stored or modified by the processors in accordance with the instructions 498. For instance, although the present disclosure is not limited by a particular data structure, data 493 may be stored in computer registers, in a relational database as a table having a plurality of different fields and records, XML documents, or flat files. Data 493 may also be formatted in a computer-readable format such as, but not limited to, binary values, ASCII or Unicode. Moreover, data 493 may comprise information sufficient to identify the relevant information, such as numbers, descriptive text, proprietary codes, pointers, references to data stored in other memories (including other network locations) or information that is used by a function to calculate the relevant data.

Instructions 498 may control various components and functions of device 400. For example, instructions 498 may be executed to selectively activate light source 410 or process information obtained by photodetector 420. In some examples, algorithms can be included as a subset of or otherwise as part of instructions 498 included in electronics 499. Instructions 498 can include algorithms to interpret or process information received, such as information received through or generated by analyzing signals or data received at sensors 310 or 315. For example, physical parameters of the user can be extracted or analyzed through algorithms. In some examples, the sensors can be configured to measure analytes of interest in the sweat collected, such as, for example, glucose, Na+, and Cl−, which can further be analyzed. In other examples, the sensors can provide data or signals related to electrical or voltage information across the skin of a user for analysis by instructions 498.

Without limitation the algorithms, whether used for ECG or other types of analysis, could use any or all information about the waveform, such as shape, frequency, or period of a wave, Fourier analysis of the signal, harmonic analysis, pulse width, pulse area, peak to peak interval, pulse interval, intensity or amount of light received by a photodetector, wavelength shift, first or second derivatives of the signal generated or received by sensors 310 or 315. Other algorithms can be included to calculate absorption of oxygen in oxyhemoglobin and deoxyhemoglobin, heart arrhythmias, heart rate, premature ventricular contractions, missed beats, systolic and diastolic peaks, large artery stiffness index, In yet other examples, artificial learning or machine learning algorithms can be used in both deterministic and non-deterministic ways to extract information related to a physical condition of a user such as blood pressure and stress levels (from heart rate variability). In some examples, machine learning based ECG algorithms can be used.

In some examples, the algorithm can include algorithms to perform PPG. PPG can also be used to measure blood pressure by computing the pulse wave velocity between two points on the skin separated by a certain distance. Pulse wave velocity is proportional to blood pressure and that relationship can be used to calculate the blood pressure.

In some examples, the algorithms can be modified or use information input by a user into memory of electronics 499 such as the user's weight, height, age, cholesterol, genetic information, body fat percentage, or other physical parameter. In other examples, machine learning algorithms can be used to detect and monitor for known or undetected health conditions, such as an arrhythmia, based on information generated by the photodetectors and/or processors.

User interface 494 may be a screen which allows a user to interact with device 400, such as a touch screen or buttons. Display 495 can be an LCD, LED, mobile phone display, electronic ink, or other display to display information about device 400. User interface 494 can allow for both input from a user and output to a user. Communication interface(s) 497 can include hardware and software to enable communication of data over standards such as Wi-Fi, Bluetooth, infrared, radio-wave, and/or other analog and digital communication standards. Communication interface(s) 497 allow for electronics 399 to be updated and information generated by device 400 to be shared to other devices. In some examples, communication interface(s) 497 can send historical information stored in memory 492 to another user device for display, storage, or further analysis. In other examples, communication interface(s) 497 can send the signal generated by the photodetector to another user device in real-time or afterwards for display on that device.

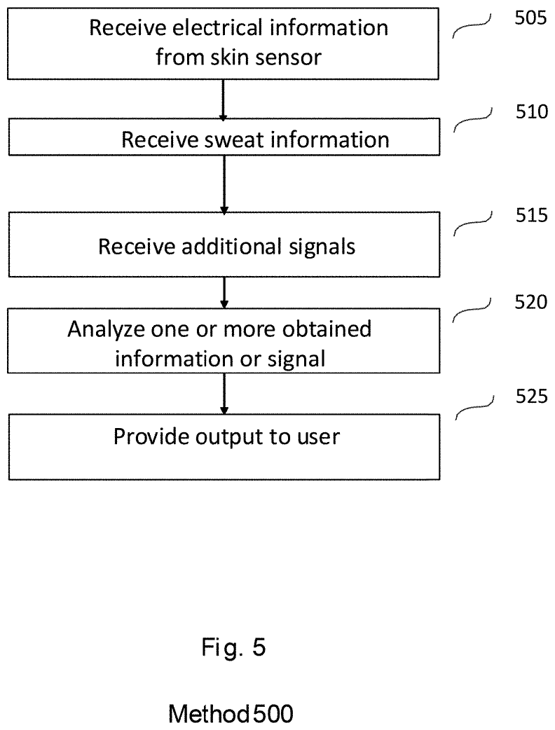

FIG. 5 illustrates a flowchart of an example method 500 of monitoring a physical parameter of a user, such as ECG information, cardiac information, or parameters detectable through analysis of sweat.

At block 505, sensor data is received. The sensor data can be received from one or more sensors, such as sensor 310 or sensor 315. The one or more sensors can be coated or partially formed using carbon nanotubes. The length of the carbon nanotubes can be running in a direction perpendicular to the contact of the skin. In some examples, at block 505, only electrical information related to the skin can be obtained from a sensor configured to perform or obtain electrical information from the skin or perform ECG measurements.

At block 510, similar to block 505, sweat related information can be obtained. The sweat related information can be received by one or more sensors, such as sensor 310 or sensor 315, which can also be coated or partially formed using carbon nanotubes. In some examples, the sweat can be wicked to a sweat-sensing sensor through the carbon nanotube. The sweat can contain biological markers or other metabolites or analytes which can be sensed by the sensor. In some examples, the sweat related information can be obtained from a sensor or electrode specifically

At block 515, additional signals or data can be obtained from additional sensors on a user device, such as for example, gyroscopes or ambient light sensors. This can include information added or provided by a user or information received over a communication interface, such as communication interface 497. In some examples, other sensors or processors can provide additional information or data, such as rotation or gyroscope information, presence of an electrical field, PPG information, ambient light information, proximity or touch information. In some examples, the information

At block 520, the information or signals obtained from blocks 505 to 515 can be analyzed or processed using ECG or other analysis algorithms. Non-limiting examples of analysis algorithms include ECG algorithms, sweat constituency algorithms, PPG analysis algorithms, or other algorithms described herein. In some examples, the information can include analysis of ECG information.

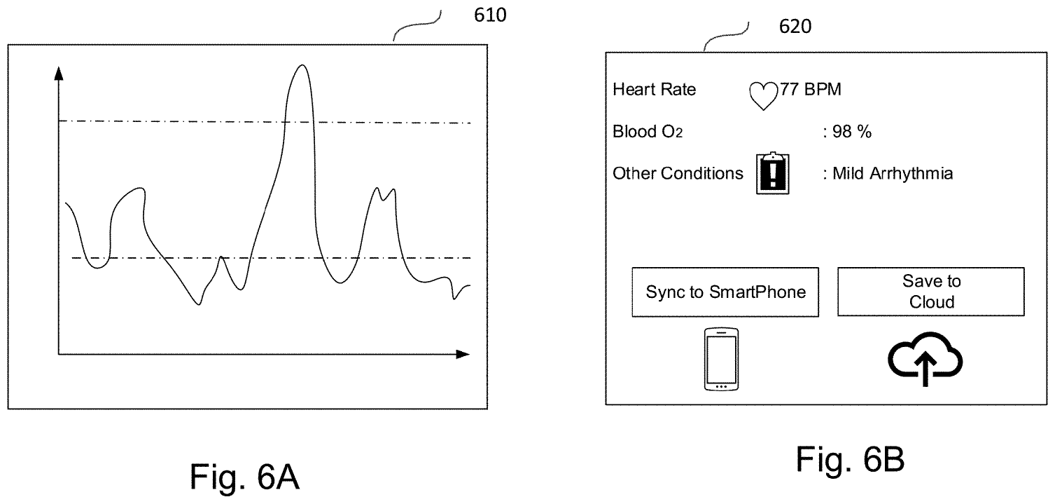

At block 525, an output can be provided to a user. In some examples, the output can be summary information which can be provided to the user through a user interface, such as interface 494 or display 203. Example displays of information are provided in FIGS. 6A and 6B.

While the method 500 is described below in a particular order, it should be understood that the operations may be performed in a different order or simultaneously. Moreover, operations may be added or omitted.

FIGS. 6A and 6B illustrate example displays of summary information, displays 610 and 620 respectively. Display 610 illustrates a graph over time of cardiac activity of a user. Display 620 displays a graph of various cardiac information for a user, including heart rate, any arrhythmia conditions, and blood oxygen levels.

While this disclosure contains many specific implementation details, these should not be construed as limitations on the scope of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

1. A wearable device comprising:
a first back surface with a substantially lateral plane;
carbon nanotubes extending substantially orthogonally to the first back surface;
a memory containing instructions, the instructions configured to analyze health signals received or obtained by the wearable device;
a processor to analyze the health signals and provide summary information; and
a display to display the summary information.

2. The device of claim 1 further comprising a health sensor located parallel to the lateral plane of the first back surface to analyze health information of a user.

3. The device of claim 2 wherein the health sensor is an ECG electrode.

4. The device of claim 3 wherein the carbon fibers extend lengthwise orthogonally to the ECG electrode.

5. The device of claim 4 wherein the carbon fibers are configured to wick sweat from the skin of a user towards the ECG electrode through capillary action.

6. The device of claim 3 further comprising a sweat sensor, the sweat sensor configured to analyze analytes or metabolites contained within the sweat of a user.

7. The device of claim 6 wherein the carbon fibers extend lengthwise orthogonally to the sweat sensor.

8. The device of claim 7 wherein the carbon fibers are configured to wick swat from the skin of a user towards the sweat sensor.

9. The device of claim 4 wherein an ECG algorithm is used to analyze the ECG signals.

10. The device of claim 4 wherein the ECG algorithm is a machine learning algorithm.

11. The device of claim 5 wherein the density of the carbon fibers is selected to increase the wicking of sweat.

12. The device of claim 5 wherein the radius of each carbon fiber from the carbon fibers is selected to increase the wicking of sweat or a capillary action.

13. The device of claim 5 wherein the relative height and radius of the carbon fiber from the carbon fibers is selected to increase the wicking of sweat.

14. The device of claim 6 wherein the density of the carbon fibers is selected to increase the wicking of sweat.

15. The device of claim 6 wherein the radius of the carbon fiber is selected to increase the wicking of sweat or a capillary action.

16. The device of claim 6 wherein the relative height and radius of the carbon fiber from the carbon fibers is selected to increase the wicking of sweat.

17. A method comprising:
receiving an ECG signal from an ECG electrode, the ECG electrode configured to contact skin of a user, and the ECG electrode containing carbon fibers extending outwards from the ECG electrode;
analyzing the ECG signal to determine health information of the user; and
displaying, to the user, health information obtained from the analyzed ECG signal;
wherein the ECG signal has been enhanced due to the presence of carbon fibers.

18. The method of claim 17 wherein the health information is analyzed using a machine learning algorithm.

19. The method of claim 17 wherein the ECG signal is analyzed with an ECG algorithm.

20. A non-transitory computer readable medium containing instructions, the instructions when executed configured to
receive an ECG signal from an ECG electrode, the ECG electrode configured to contact skin of a user, and the ECG electrode containing carbon fibers extending outwards from the ECG electrode;
analyze the ECG signal to determine health information of the user; and
display, to the user, health information obtained from the analyzed ECG signal;
wherein the ECG signal has been enhanced due to the presence of carbon fibers.
